(19) United States
(12) Patent Application Publication
(10) Pub. No.: US 2006/0040366 A1
(43) Pub. Date: Feb. 23, 2006

(54) New polyhydroxyalkanoate comprising unit having (phenylmethyl) oxy structure on side chain thereof, and method for producing the same

(75) Inventors: Takashi Kenmoku, Tokyo (JP); Tetsuya Yano, Tokyo (JP); Shinya Kozaki, Tokyo (JP); Tsutomu Honma, Tokyo (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 10/531,572

(22) PCT Filed: Oct. 23, 2003
(86) PCT No.: PCT/JP03/13530

§ 371 Date: Apr. 18, 2005

(30) Foreign Application Priority Data
Oct. 24, 2002 (JP) NO. 2002-309786
Oct. 16, 2003 (JP) NO. 2003-356749

(51) Int. Cl. C12P 7/62; C08G 69/44

(52) U.S. Cl. 435/135; 528/288

(57) ABSTRACT

Provided are a PHA having an active group, which is produced by microorganisms with high productivity, and in which the ratio of units on its side chain having an active group can be controlled and the physical properties can be arbitrarily controlled so that its application as a polymer is not limited, and a method for producing the same.
A 3-hydroxy-ω-[(phenylmethyl)oxy]alkanoic acid unit expressed by the following chemical formula (1): 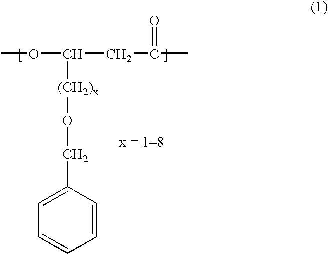 wherein x can be one or more integers within the range shown in the chemical formula.

### TECHNICAL FIELD

The present invention relates to a polyhydroxyalkanoate comprising a new unit, and a method for producing the same using microorganisms. Hereinafter, the term “polyhydroxyalkanoate” is referred to as “PHA” at times.

### BACKGROUND ART

It has been reported so far that many types of microorganisms produce poly-3-hydroxybutyric acid (PHB) or other polyhydroxyalkanoates (PHAs) and accumulate them in the cells. As with the conventional plastics, polymers such as polyhydroxyalkanoate produced by microorganisms are subjected to melting processing, so that they can be used for production of various types of products. Moreover, polymers produced by microorganisms, such as polyhydroxyalkanoates, are biodegradable, and accordingly, they have an advantage in that they are completely decomposed by microorganisms existing in the nature. Accordingly, for example, when a polyhydroxyalkanoate produced by microorganisms is discarded, differing from many conventional synthetic polymer compounds, it does not remain in the environment as is, and therefore it does not cause pollution. Furthermore, since polyhydroxyalkanoate produced by microorganisms have excellent biocompatibility, it is expected that these compounds will be applied to soft components for medical use, etc.

It is known that these polyhydroxyalkanoates produced by microorganisms can have various compositions or structures depending on the type of microorganisms used for production, the composition of a medium, culture conditions, etc. Studies have been made so far to attempt to control the composition or structure of the polyhydroxyalkanoate produced by microorganisms, mainly from the viewpoint of the improvement of physical properties of the polyhydroxyalkanoate.

As a study directed towards controlling the composition or structure of the polyhydroxyalkanoate produced by microorganisms, a study has been vigorously made to allow microorganisms to produce polyhydroxyalkanoate having an aromatic ring in its unit in these years.

(a) Polyhydroxyalkanoate Comprising Phenyl Group or its Partially Substituted Form

It has been reported that using 5-phenyl valeric acid as a substrate, Pseudomonas oleovorans produces a polyhydroxyalkanoate comprising 3-hydroxy-5-phenyl valeric acid as a unit (Makromol. Chem., Vol. 191, 1990, pp. 1957-1965, Macromolecules, Vol. 24, 1991, pp. 5256-5260).

It has been reported that using 5-(p-tolyl)valeric acid as a substrate, Pseudomonas oleovorans produces a polyhydroxyalkanoate comprising 3-hydroxy-5-(p-tolyl)valeric acid as a unit (Macromolecules, Vol. 29, 1996, pp. 1762-1766).

It has been reported that using 5-(2,4-dinitrophenyl)valeric acid as a substrate, Pseudomonas oleovorans produces a polyhydroxyalkanoate comprising 3-hydroxy-5-(2,4-dinitrophenyl)valeric acid and 3-hydroxy-5-(p-nitrophenyl)valeric acid as units (Macromolecules, Vol. 32, 1999, pp. 2889-2895).

(b) Polyhydroxyalkanoate Comprising Phenoxy Group or its Partially Substituted Form

It has been reported that using 11-phenoxy undecanoic acid as a substrate, Pseudomonas oleovorans produces a polyhydroxyalkanoate copolymer comprising a 3-hydroxy-5-phenoxy valeric acid unit and a 3-hydroxy-9-phenoxynonanoic acid unit (Macromol. Chem. Phys., Vol. 195, 1994, pp. 1665-1672).

There has been disclosed an invention relating to a homopolymer consisting of 3-hydroxy-5-(monofluorophenoxy)pentanoate (3H5(MFP)P) units or 3-hydroxy-5-(difluorophenoxyl)pentanoate (3H5(DFP)P) units; a copolymer containing at least (3H5(MFP)P) units or (3H5(DFP)P) units; Pseudomonas putida having an ability to synthesize these polymers; and a method of producing the above described polymers using Pseudomonas species. In addition, it is described as an advantage of the above invention that a long chain aliphatic acid having substituent groups can be assimilated to synthesize a polymer having a phenoxy group substituted with 1 or 2 fluorine atoms at the side chain terminal and that the polymer provides stereoregularity and water repellency, while maintaining a high melting point and good processability (Japanese Patent No. 2989175).

Moreover, studies are conducted on a polyhydroxyalkanoate in which a cyano or nitro group is substituted on an aromatic ring in its unit, as well as on fluorine-substituted PHA in which fluorine is substituted on an aromatic ring in its unit.

It has been reported that a polyhydroxyalkanoate containing 3-hydroxy-6-(p-cyanophenoxy)hexanoic acid or 3-hydroxy-6-(p-nitrophenoxy)hexanoic acid as a monomer unit is produced with octanoic acid and 6-(p-dyanophenoxy)hexanoic acid or 6-(p-nitrophenoxy)hexanoic acid as substrates, using a Pseudomonas oleovorans ATCC 29347 strain and a Pseudomonas putida KT 2442 strain (Can. J. Microbiol, Vol. 41, 1995, pp. 32-43 and Polymer International, Vol. 39, 1996, pp. 205-213).

Such a polyhydroxyalkanoate containing units each having an aromatic ring having a substituent group thereof can be a multifunctional polyhydroxyalkanoate, which possesses a new function derived from the substituent group existing on the aromatic ring, while maintaining polymer characteristics derived from the aromatic ring, such as a high glass transition temperature and good processability.

At the same time, studies are vigorously conducted directed towards the obtainment of a multifunctional polyhydroxyalkanoate, which is based on a polyhydroxyalkanoate having a bromo group in its unit and obtained by introducing any given functional group into the side chain of a produced polymer by chemical transformation using the above bromo group.

It has been reported that a polyhydroxyalkanoate having a bromo group on a side chain thereof is produced using Pseudomonas oleovorans, and then the side chain is modified with the thiolated product of an acetylated maltose, to synthesize a polyhydroxyalkanoate having different solubility and hydrophilicity (Macromol. RapidCommun., Vol. 20, 1999, pp. 91-94).

It has been reported that polyester having a vinyl group on a side chain thereof is produced using Pseudomonas oleovolans, and then the vinyl group in the polyester molecule is oxidized, so as to produce polyester having an epoxy group on its side chain (Polymer, Vol. 41, 2000, pp. 1703-1709).

It has been reported that polyester having a vinyl group on a side chain thereof is produced using Pseudomonas oleovolans, and then the vinyl group is epoxidized, to produce polyester having an epoxy group on its side chain (Macromolecules, Vol. 31, 1998, pp. 1480-1486).

It has been reported that using a vinyl group on the side chain of polyester, a crosslinking reaction is carried out in the polyester molecule, to improve the properties of the polyester (Polymer, Vol. 35, 1994, pp. 2090-2097).

To change the physical properties of a PHA having an active group in its unit to actually use it as a polymer, the synthesis of a PHA copolymer comprising units other than units having active groups by using microorganisms has been studied. It has been reported that using Pseudomonas oleovorans, a PHA copolymer comprising a 3-hydroxy-ω-bromoalkanoic acid unit and a straight-chain alkanoic acid unit has been produced in the coexistence of ω-bromoalkanoic acid such as 11-bromoundecanoic acid, 8-bromooctanoic acid and 6-bromohexanoic acid and n-nonanoic acid (Macromolecules, Vol. 25, 1992, pp. 1852-1857).

Thus, into PHA having an active group with high reactivity, such as a bromo or vinyl group, in its units, various functional groups can be introduced. Or, chemical transformation can also be performed on such PHA. Moreover, since PHA having an active group can be a crosslink point of a polymer, it can be said that such PHA is extremely effective for achievement of multifunctional PHA.

However, in a case where a PHA having a bromo group as an active group is synthesized using microorganisms, the productivity of the obtained PHA is low. In a case where a PHA copolymer is synthesized using microorganisms, it is difficult to increase or control the unit ratio of bromo groups.

Further, in the case of the synthesis of a PHA having a vinyl group as an active group, if the vinyl group is located at the end of an alkyl chain, the synthesized PHA has a low glass transition temperature and a low melting point, and therefore it cannot be said that the obtained PHA has physical properties preferable for the processability and usability of the polymer.

For the above described reasons, a new PHA having an active group, which is produced by microorganisms with high productivity, and in which the ratio of units in its side chain having an active group can be controlled and the physical properties can be arbitrarily controlled so that its application as a polymer is not limited, and a method for producing the same, have been desired.

### DISCLOSURE OF THE INVENTION

As a result of intensive studies directed towards achieving the above object, the present inventors have found a method for synthesizing a PHA comprising a unit having a highly reactive (phenylmethyl)oxy structure as an active group by using microorganisms, thereby completing the present invention.

According to an aspect of the present invention, there is provided a polyhydroxyalkanoate comprising a 3-hydroxy-ω-[(phenylmethyl)oxy]alkanoic acid unit expressed by chemical formula (1): 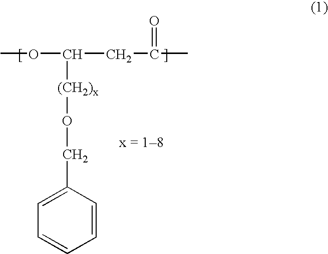
wherein x can be one or more integers within the range shown in the chemical formula.

According to another aspect of the present invention, there is provided a method for producing a polyhydroxyalkanoate comprising, in a molecule thereof, a 3-hydroxy-ω-[(phenylmethyl)oxy]alkanoic acid unit expressed by chemical formula (1), which comprises allowing a microorganism with an ability to produce a polyhydroxyalkanoate comprising in a molecule thereof a 3-hydroxy-ω-[(phenylmethyl)oxy]alkanoic acid unit expressed by chemical formula (1) to biosynthesize the polyhydroxyalkanoate under a condition which comprise ω-[(phenylmethyl)oxy]alkanoic acid expressed by chemical formula (19): 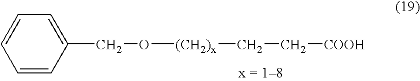
wherein x can be one or more integers within the range shown in the chemical formula.

### BRIEF DESCRIPTION OF THE DRAWINGS

FIG. 1 is a ¹H-NMR spectrum chart of the polyhydroxyalkanoate in Example 1; and

FIG. 2 is a ¹H-NMR spectrum chart of the polyester obtained in Example 23.

### BEST MODE FOR CARRYING OUT THE INVENTION

The method for producing the polyhydroxyalkanoate of the present invention comprises culturing microorganism in a medium containing at least one selected from a group consisting of peptides, yeast extract, organic acids or salts thereof, amino acids or salts thereof, saccharides, straight-chain alkanoic acids, which is saturated or unsaturated fatty acid having 4 to 12 carbon atoms or salts thereof, in addition to the ω-[(phenylmethyl)oxy]alkanoic acid expressed by the above chemical formula (19). Moreover, in the above culture of microorganisms, the peptide contained in the medium is polypeptone; the organic acids or salts thereof contained in the medium are one or more compounds selected from a group consisting of pyruvic acid, oxaloacetic acid, citric acid, isocitric acid, ketoglutaric acid, succinic acid, fumaric acid, malic acid, lactic acid, and salts thereof; the amino acids or salts thereof contained in the medium are one or more compounds selected from a group consisting of glutamic acid, asparaginic acid, and salts thereof; and the saccharides contained in the medium are one or more compounds selected from a group consisting of glyceraldehyde, erythrose, arabinose, xylose, glucose, galactose, mannose, fructose, glycerol, erythritol, xylitol, gluconic acid, glucuronic acid and galacturonic acid, maltose, sucrose and lactose.

The polyhydroxyalkanoate of the present invention preferably further comprises at least one unit expressed by chemical formula selected from the group consisting of chemical formulas (2) and (3): 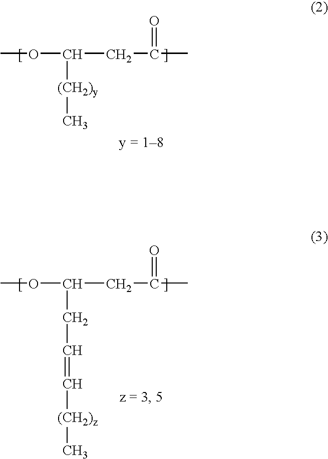
wherein y and z can be one or more integers within the range shown in the chemical formulas, while being independent from the unit expressed by chemical formula (1).

Alternatively, the PHA of the present invention preferably comprises, in a molecule thereof, the 3-hydroxy-ω-[(phenylmethyl)oxy]alkanoic acid unit expressed by chemical formula (1) and a 3-hydroxy-alkanoic acid unit expressed by chemical formula (4): 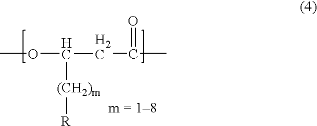
wherein m can be one or more integers within the range shown in the chemical formula, and R comprises a residue having either a phenyl structure or thienyl structure, or a 3-hydroxy-ω-cyclohexylalkanoic acid unit expressed by chemical formula (5): 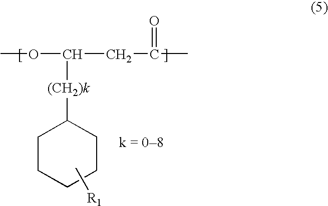
wherein R₁ is H, CN, NO₂, halogen, CH₃, C₂H₅, C₃H₇, CF₃, C₂F₅ and C₃F₇, and k can be one or more integers within the range shown in the chemical formula. R in chemical formula (4) is preferably a group selected from the group consisting of 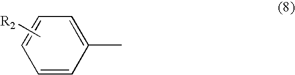
wherein R₂ is H, halogen, CN, NO₂, CH₃, C₂H₅, C₃H₇, CH═CH₂, COOR₃ (wherein R₃ represents any one selected from the group consisting of H, Na and K), CF₃, C₂F₅ and C₃F₇, and in a case where there exist a plurality of units, R₂ may be different for each unit; 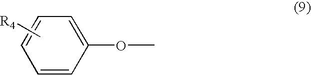
wherein R₄ is selected from the group consisting of H, halogen, CN, NO₂, CH₃, C₂H₅, C₃H₇, SCH₃, CF₃, C₂F₅ and C₃F₇, and in a case where there exist a plurality of units, R₄ may be different for each unit; 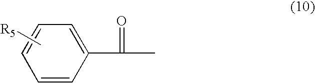
wherein R₅ is selected from the group consisting of H, halogen, CN, NO₂, CH₃, C₂H₅, C₃H₇, CF₃, C₂F₅ and C₃F₇, and in a case where there exist a plurality of units, R₅ may be different for each unit; 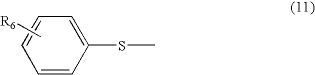
wherein R₆ is selected from the group consisting of H, halogen, CN, NO₂, COOR₇, SO₂R₈ (wherein R₇ represents any one selected from the group consisting of H, Na, K, CH₃ and C₂H₅, and R₈ represents any one selected from the group consisting of OH, ONa, OK, halogen, OCH₃ and OC₂H₅), CH₃, C₂H₅, C₃H₇, (CH₃)₂—CH, and (CH₃)₃—C, and in a case where there exist a plurality of units, R₆ may be different for each unit; 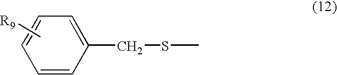
wherein R₉ represents a substituent group on the aromatic ring, R₉ is selected from thg group consisting of H, halogen, CN, NO₂, COOR₁₀, SO₂R₁₁ (wherein R₁₀ represents any one selected from the group consisting of H, Na, K, CH₃ and C₂H₅, and R₁, represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₉ may be different for each unit; 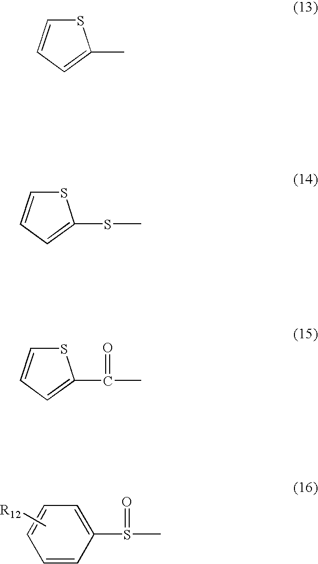
wherein R₁₂ is selected from thg group consisting of H, halogen, CN, NO₂, COOR₁₃, SO₂R₁₄ (wherein R₁₃ represents any one selected from the group consisting of H, Na, K, CH₃ and C₂H₅, and R₁₄ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₁₂ may be different for each unit; and 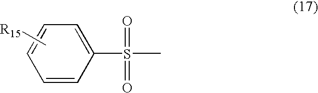
wherein R₁₅ is selected from the group consisting of H, halogen, CN, NO₂, COOR₁₆, SO₂R₁₇ (wherein R₁₆ represents any one selected from the group consisting of H, Na, K, CH₃ and C₂H₅, and R₁₇ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₁₅ may be different for each unit.

In the PHA of the present invention, the 3-hydroxy-ω-[(phenylmethyl)oxy]alkanoic acid unit expressed by chemical formula (1) is preferably either one or both of:
a 3-hydroxy-4-[(phenylmethyl)oxy]butyric acid unit expressed by chemical formula (6): 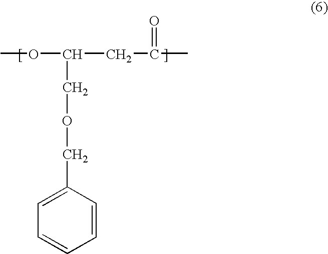
   and a 3-hydroxy-5-[(phenylmethyl)oxy]valeric acid unit expressed by chemical formula (7):

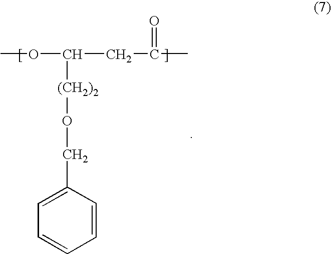

In the PHA of the present invention, a number average molecular weight is preferably within the range between 1,000 and 1,000,000.

In the method for producing a polyhydroxyalkanoate of the present invention, the polyhydroxyalkanoate preferably comprises at least one unit expressed by chemical formulas (2) and (3).

In the method for producing a polyhydroxyalkanoate of the present invention, the ω-[(phenylmethyl)oxy]alkanoic acid expressed by the chemical formula (19) is preferably 4-[(phenylmethyl)oxy]butyric acid expressed by chemical formula (23): 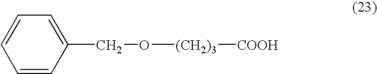
or 5-[(phenylmethyl)oxy]valeric acid expressed by chemical formula (24):

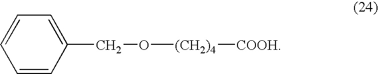

The method for producing a polyhydroxyalkanoate of the present invention may comprise allowing the microorganism with an ability to produce a polyhydroxyalkanoate comprising, in a molecule thereof, the 3-hydroxy-ω-[(phenylmethyl)oxy]alkanoic acid unit expressed by chemical formula (1) and
a 3-hydroxy-alkanoic acid unit expressed by chemical formula (22): 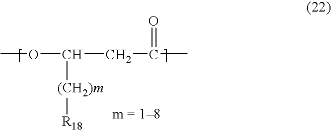
   wherein m can be one or more integers within the range shown in the chemical formula, and R₁₈ comprises a residue having either a phenyl structure or thienyl structure, or a 3-hydroxy-ω-cyclohexylalkanoic acid unit expressed by chemical formula (5), from ω-[(phenylmethyl)oxy]alkanoic acid expressed by chemical formula (19), and alkanoic acid expressed by chemical formula (20): 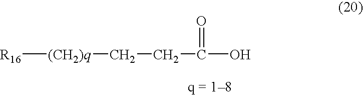
   wherein q can be one or more integers within the range shown in the chemical formula, and R₁₆ comprises a residue having either a phenyl structure or thienyl structure
or ω-cyclohexylalkanoic acid expressed by chemical formula (21) 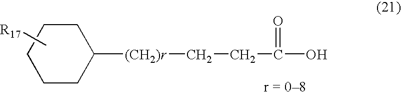
   wherein R₁₇ is selected from the group consisting of H, CN, NO₂, halogen, CH₃, C₂H₅, C₃H₇, CF₃, C₂F₅ and C₃F₇, and r can be one or more integers within the range shown in the chemical formula as raw materials to biosynthesize the polyhydroxyalkanoate under a condition which comprise ω-[(phenylmethyl)oxy]alkanoic acid expressed by chemical formula (19), and alkanoic acid expressed by chemical formula (20) or a ω-cyclohexylalkanoic acid expressed by chemical formula (21). In particular, R₁₆ in chemical formula (20) and R₁₈ in chemical formula (22) are preferably groups independently selected from the group consisting of chemical formula (25): 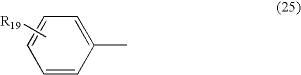
   wherein R₁₉ is selected from the group consisting of H, halogen, CN, NO₂, CH₃, C₂H₅, C₃H₇, CH═CH₂, CF₃, C₂F₅ and C₃F₇, and in a case where there exist a plurality of units, R₁₉ may be different for each unit; chemical formulae (9), (10), (11), (12), (13), (14), (15), (16) and (17). Alternatively, the condition is preferably that the microorganism is cultured in a medium containing the ω-[(phenylmethyl)oxy]alkanoic acid expressed by chemical formula (19) and the alkanoic acid expressed by chemical formula (20) or the α-cyclohexylalkanoic acid expressed by chemical formula (21).

In the method for producing a polyhydroxyalkanoate of the present invention, the condition is preferably that the microorganisms is cultured in a medium containing ω-[(phenylmethyl)oxy]alkanoic acid expressed by chemical formula (19). In particular, the medium preferably contains at least one selected from the group consisting of peptides, yeast extract, organic acids or salts thereof, amino acids or salts thereof, saccharides and straight-chain alkanoic acids, which is saturated or unsaturated fatty acid having 4 to 12 carbon atoms or salts thereof. More especially, the peptide is polypeptone; the organic acids or salts thereof are one or more compounds selected from the group consisting of pyruvic acid, oxaloacetic acid, citric acid, isocitric acid, ketoglutaric acid, succinic acid, fumaric acid, malic acid, lactic acid, and salts thereof; the amino acids or salts thereof are one or more compounds selected from the group consisting of glutamic acid, aspartic acid, and salts thereof; and the saccharides are one or more compounds selected from the group consisting of glyceroaldehyde, erythrose, arabinose, xylose, glucose, galactose, mannose, fructose, glycerol, erythritol, xylitol, gluconic acid, glucuronic acid and galacturonic acid, maltose, sucrose and lactose.

When cultivating the microorganism in the medium containing the alkanoic acid expressed by chemical formula (19) in the method for producing a polyhydroxyalkanoate of the present invention, the culture of microorganisms preferably comprises two or more culturing steps. In particular, the culture is preferably a fed-batch culture.

When cultivating the microorganism in the medium containing the alkanoic acid expressed by chemical formula (19) in the method for producing a polyhydroxyalkanoate of the present invention as mentioned above, the method preferably comprises a step of recovering a polyhydroxyalkanoate comprising 3-hydroxy-ω-[(phenylmethyl)oxy]alkanoic acid unit expressed by chemical formula (1) generated by the microorganism from the cells of the microorganism. In the method for producing a polyhydroxyalkanoate of the present invention, the microorganism preferably belongs to Pseudomonas species. In particular, the microorganism is preferably one or more strains selected from the group consisting of Pseudomonas cichorii YN2 (FERM BP-7375), Pseudomonas cichorii H45 (FERM BP-7374) and Pseudomonas jessenii P161 (FERM BP-7376).

Detailed culture conditions of microorganisms in the method for producing the polyhydroxyalkanoate of the present invention are as follows:

As described below, various necessary substrates and nutrients are added to an inorganic salt medium basically containing a phosphate buffer and ammonium salts or nitrates.

In order to produce a polyhydroxyalkanoate of interest expressed by the above chemical formula (1) comprising a 3-hydroxy-ω-(phenylmethyl)alkanoic acid unit, it is desirable that ω-[(phenylmethyl)oxy]alkanoic acid expressed by the above chemical formula (19) is contained in the medium as a substrate, at a proportion from 0.01% to 1% (w/v) per medium, and more preferably at a proportion from 0.02% to 0.2% per medium.

In order to produce a polyhydroxyalkanoate comprising in a molecule thereof a 3-hydroxy-alkanoic acid unit expressed by chemical formula (22) or a 3-hydroxy-ω-cyclohexylalkanoic acid unit expressed by chemical formula (5) as well as a 3-hydroxy-ω-[(phenylmethyl)oxy]alkanoic acid unit, it is desirable that each of ω-[(phenylmethyl)oxy]alkanoic acid of the above chemical formula (19) and an alkanoic acid expressed by chemical formula (20) or ω-cyclohexylalkanoic acid expressed by the above chemical formula (21) is contained as a substrate in the medium at a proportion from 0.01% to 1% (w/v) per medium, and more preferably at a proportion from 0.02% to 0.2% per medium.

A carbon source and a nitrogen source for growth of microorganisms and for production of a polyhydroxyalkanoate are preferably added to the medium at a concentration from 0.1% to 5% (w/v) per medium, and more preferably from 0.2% to 2% per medium.

Any medium can be used in the present invention, as long as it is an organic salt medium containing phosphate and a nitrogen source such as ammonium salts or nitrates. It is possible to increase the productivity of PHA by controlling the concentration of the nitrogen source.

Any temperature is applied as a culture temperature, as long as the above cell strains can grow favorably at the temperature. A temperature between 15° C. and 37° C. is appropriate, and a temperature between 20° C. and 30° C. is more preferable.

Any culture method can be used, as long as microorganisms can grow and produce PHA. Not only one stage culture such as usual batch culture but also two stage culture comprised of the steps of collecting once cells obtained by a one stage culture and adding the collected cells to another new medium to carry out a cultivation again can be also employed. A fed-batch culture, in which a new medium is added to the culture liquid without collecting the cells, can be used so as to carry out the two stage culture more simply. A continuous culture also can be used. Further, any form of the culture can be used, including a method of shaking a medium in a vessel for cultivation such as flask, a method with a fermenter and so forth.

In addition to the above described methods, there is another method for allowing microorganisms to produce and accumulate PHA. This method comprises making microorganism sufficiently grow, transferring the cells to a medium in which a nitrogen source such as ammonium chloride is limited, and further culturing them in a state where a compound as a substrate of a unit of interest is present. This method improves productivity in some cases.

Moreover, after the microorganisms are cultured under the above described conditions, the method may comprise a step of recovering a polyhydroxyalkanoate comprising the 3-hydroxy-ω-[(phenylmethyl)oxy]alkanoic acid unit expressed by the above chemical formula (1) produced by the above microorganisms from the cells.

As a method of recovering a PHA of interest from the cells of microorganisms, a common method can be adopted. For example, extraction with an organic solvent such as chloroform, dichloromethane, ethylacetate or acetone is the most simple, but dioxane, tetrahydrofuran or acetonitrile may also be used in some cases. In an environment in which the use of organic solvents is not preferred, any one of a treatment with surfactants such as SDS, a treatment with an enzyme such as lysozyme, a treatment with chemicals such as hypochlorite, ammonium or EDTA, an ultrasonic crushing method, a homogenizer method, a pressure crushing method, a bead impulse method, a grinding method, an immersion method and a freeze-thaw method may be used to physically crush microorganism cells. Cell components other than PHA are removed by any one of the above methods, to collect PHA.

As microorganisms used for the production method of the present invention, any species of microorganisms may be used, as long as they have an ability to satisfy the above described conditions. Among them, microorganisms belonging to Pseudomonas species are desirable. Specific examples of preferred species include Pseudomonas cichorii, Pseudomonas putida, Pseudomonas fluorecense, Pseudomonas oleovorans, Pseudomonas aeruginosa, Pseudomonas stutzeri, and Pseudomonas jessenii. More specifically, examples of a suitable strain include Pseudomonas cichorii YN2 (FERM BP-7375), Pseudomonas cichorii H45 (FERM BP-7374), and Pseudomonas jessenii P161 (FERM BP-7376). These three types of strains were deposited on Nov. 20, 2000 at the International Patent Organism Depositary (IPOD) of National Institute of Advanced Industrial Science and Technology (AIST), Tsukuba Central 6, 1-1, Higashi 1-chome, Tsukuba-shi, Ibaraki-ken 305-8566, Japan, and they are described in U.S. Pat. No. 6,586,562.

It should be noted that the culture of microorganisms in the present invention, the production of PHA by microorganisms and accumulation in the cells in the present invention, and the recovery of PHA from the cells in the present invention are not limited to the above described methods.

The composition of an inorganic salt culture medium (M9 medium) used in one method of the present invention is shown below.

[M9 medium]

Na₂HPO₄: 6.3
KH₂PO₄: 3.0
NH₄Cl: 1.0
NaCl: 0.5 g/L, pH 7.0

To ensure satisfactory growth of cells and associated good productivity of PHA, it is necessary to add an approximately 0.3% (v/v) trace component solution shown below to the above inorganic salt medium.

[Trace Component Solution]

nitrilotriacetic acid: 1.5; MgSO₄: 3.0; MnSO₄: 0.5;
NaCl: 1.0; FeSO₄: 0.1;
CaCl₂: 0.1; CoCl₂: 0.1; ZnSO₄: 0.1; CuSO₄: 0.1;
AlK(SO₄)₂: 0.1, H₃BO₃: 0.1; Na₂MoO₄: 0.1; NiCl₂: 0.1 g/L

### EXAMPLES

### Example 1

A Pseudomonas cichorii YN2 strain was inoculated into 200 mL of M9 medium containing 0.5% D-glucose, 0.1% polypeptone and 0.1% 5-[(phenylmethyl)oxy]valeric acid, followed by shaking the medium at 30° C. at 125 strokes/minute. At 48 hours later, cells were recovered by centrifugation, and they were resuspended in 200 mL of M9 medium containing 0.5% D-glucose and 0.1% 5-[(phenylmethyl)oxy]valeric acid, followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 33 mg of PHA.

An NMR analysis was carried out on the obtained PHA under the following conditions:
<Measuring equipment> FT-NMR: Bruker DPX400
Resonance frequency: ¹H=400 MHz
<Measuring equipment> Type of nuclear species: ¹H
Used solvent: CDCl₃
Measuring temperature: room temperature

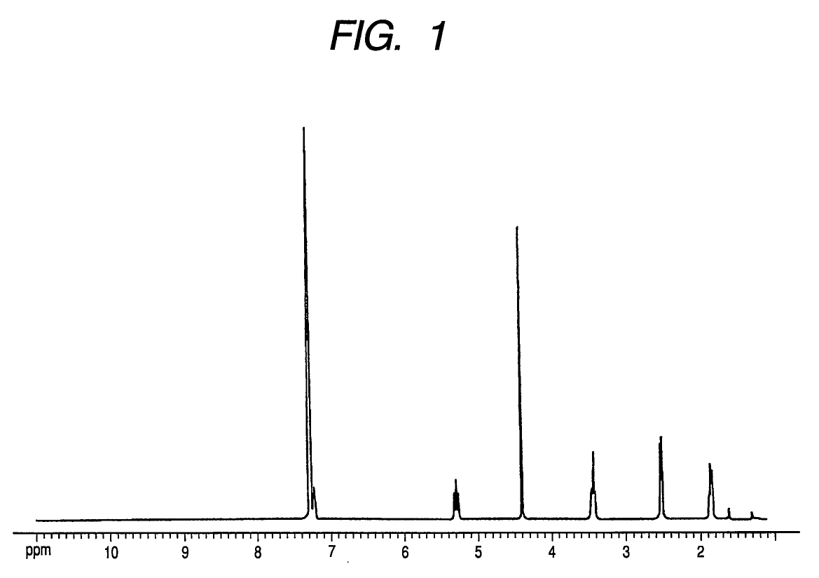

FIG. 1 shows a ¹H-NMR spectrum chart, and Table 1 shows the identification results.

TABLE 1

| Chemical shift | | | Integration |
|---|---|---|---|
| (ppm) | Attribution | Fragmentation | ratio |
| 1.86 | d1 | m | 2H |
| 2.54 | b1 | m | 2H |
| 3.44 | e1 | m | 2H |
| 4.41 | f1 | s | 2H |
| 5.31 | c1 | m | 1H |
| 7.20 to 7.31 | h1 i1 j1 k1 l1 | m | 5H |

As shown in Table 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 94.9 mol % 3-hydroxy-5-[(phenylmethyl)oxy]valeric acid as a monomer unit.

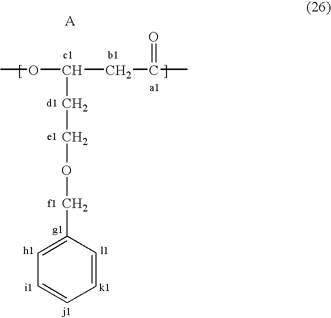

Furthermore, the molecular weight of the obtained PHA was evaluated by gel permeation chromatography (GPC: Tosoh HLC-8220, column: Tosoh TSK-GEL Super HM-H, solvent: chloroform, polystyrene conversion). As a result, Mn=123,000, and Mw=293,000.

### Example 2

A Pseudomonas cichorii YN2 strain was inoculated into 200 mL of M9 medium containing 0.5% D-glucose and 0.1% 5-[(phenylmethyl)oxy]valeric acid, followed by shaking the medium at 30° C. at 125 strokes/minute. At 48 hours later, cells were recovered by centrifugation, and they were resuspended in 200 mL of M9 medium, which contained 0.5% D-glucose and 0.1% 5-[(phenylmethyl)oxy]valeric acid but did not contain a nitrogen source (NH₄Cl), followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 30 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 92.6 mol % 3-hydroxy-5-[(phenylmethyl)oxy]valeric acid as a monomer unit.

### Example 3

A Pseudomonas cichorii H45 strain was inoculated into 200 mL of M9 medium containing 0.5% D-glucose, 0.1% polypeptone, and 0.1% 5-[(phenylmethyl)oxy]valeric acid, followed by shaking the medium at 30° C. at 125 strokes/minute. At 48 hours later, cells were recovered by centrifugation, and they were resuspended in 200 mL of M9 medium containing 0.5% D-glucose and 0.1% 5-[(phenylmethyl)oxy]valeric acid, followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 31 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 91.6 mol % 3-hydroxy-5-[(phenylmethyl)oxy]valeric acid as a monomer unit.

### Example 4

A Pseudomonas jessenii P161 strain was inoculated into 200 mL of M9 medium containing 0.5% D-glucose, 0.1% polypeptone, and 0.1% 5-[(phenylmethyl)oxy]valeric acid, followed by shaking the medium at 30° C. at 125 strokes/minute. At 48 hours later, cells were recovered by centrifugation, and they were resuspended in 200 mL of M9 medium containing 0.5% D-glucose and 0.1% 5-[(phenylmethyl)oxy]valeric acid, followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 29 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 90.8 mol % 3-hydroxy-5-[(phenylmethyl)oxy]valeric acid as a monomer unit.

### Example 5

A Pseudomonas cichorii YN2 strain was inoculated into 200 mL of M9 medium containing 0.5% D-glucose, 0.1% polypeptone, and 0.1% 5-[(phenylmethyl)oxy]valeric acid, followed by shaking the medium at 30° C. at 125 strokes/minute. At 48 hours later, 20 mL of aqueous solution containing 5% D-glucose and 1% 5-[(phenylmethyl)oxy]valeric acid was added thereto, followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 25 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 79.7 mol % 3-hydroxy-5-[(phenylmethyl)oxy]valeric acid as a monomer unit.

### Example 6

A Pseudomonas cichorii YN2 strain was inoculated into 200 mL of M9 medium containing 0.5% polypeptone and 0.1% 5-[(phenylmethyl)oxy]valeric acid, followed by shaking the medium at 30° C. at 125 strokes/minute. At 48 hours later, cells were recovered by centrifugation, and they were resuspended in 200 mL of M9 medium, which contained 0.5% pyruvic acid and 0.1% 5-[(phenylmethyl)oxy]valeric acid but did not contain a nitrogen source (NH₄Cl), followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 24 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 77.8 mol % 3-hydroxy-5-[(phenylmethyl)oxy]valeric acid as a monomer unit.

### Example 7

A Pseudomonas cichorii YN2 strain was inoculated into 200 mL of M9 medium containing 0.5% polypeptone and 0.1% 5-[(phenylmethyl)oxy]valeric acid, followed by shaking the medium at 30° C. at 125 strokes/minute. At 48 hours later, fungus cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 20 mg of PHA. As a result of carrying out an NMR analysis under the same conditions as in Example 1, it was found that the obtained PHA was a PHA expressed by chemical formula (26), which comprised 3-hydroxy-5[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 74.2 mol % 3-hydroxy-5-[(phenylmethyl)oxy]valeric acid as a monomer unit.

### Example 8

A Pseudomonas cichorii H45 strain was inoculated into 200 mL of M9 medium containing 0.5% yeast extract and 0.1% 5-[(phenylmethyl)oxy]valeric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 660° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 16 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 75.9 mol % 3-hydroxy-5-[(phenylmethyl)oxy]valeric acid as a monomer unit.

### Example 9

A Pseudomonas jessenii P161 strain was inoculated into 200 mL of M9 medium containing 0.5% glucose and 0.1% 5-[(phenylmethyl)oxy]valeric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 17 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 81.5 mol % 3-hydroxy-5-[(phenylmethyl)oxy]valeric acid as a monomer unit.

### Example 10

A Pseudomonas cichorii YN2 strain was inoculated into 200 mL of M9 medium containing 0.5% pyruvic acid and 0.1% 5-[(phenylmethyl)oxy]valeric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 10 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 88.5 mol % 3-hydroxy-5-[(phenylmethyl)oxy]valeric acid as a monomer unit.

### Example 11

A Pseudomonas cichorii H45 strain was inoculated into 200 mL of M9 medium containing 0.5% sodium glutamate and 0.1% 5-[(phenylmethyl)oxy]valeric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 12 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 86.3 mol % 3-hydroxy-5-[(phenylmethyl)oxy]valeric acid as a monomer unit.

### Example 12

A Pseudomonas jessenii P161 strain was inoculated into 200 mL of M9 medium containing 0.1% nonanoic acid and 0.1% 5-[(phenylmethyl)oxy]valeric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 9 mg of PHA. As a result of carrying out an NMR analysis under the same conditions as in Example 1, it was found that the obtained PHA was a PHA expressed by chemical formula (26), which comprised 3-hydroxy-5-[(phenylmethyl)oxy]valeric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 24.5 mol % 3-hydroxy-5-[(phenylmethyl)oxy]valeric acid as a monomer unit.

### Example 13

A Pseudomonas cichorii YN2 strain was inoculated into 200 mL of M9 medium containing 0.5% D-glucose, 0.1% polypeptone, and 0.1% 4-[(phenylmethyl)oxy]butyric acid, followed by shaking the medium at 30° C. at 125 strokes/minute. At 48 hours later, cells were recovered by centrifugation, and they were resuspended in 200 mL of M9 medium containing 0.5% D-glucose and 0.1% 4-[(phenylmethyl)oxy]butyric acid, followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 30 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 92.4 mol % 3-hydroxy-4-[(phenylmethyl)oxy]butyric acid as a monomer unit.

Furthermore, the molecular weight of the obtained PHA was evaluated by gel permeation chromatography (GPC: Tosoh HLC-8220, column: Tosoh TSK-GEL Super HM-H, solvent: chloroform, polystyrene conversion). As a result, Mn=138,000, and Mw 294,000.

### Example 14

A Pseudomonas cichorii YN2 strain was inoculated into 200 mL of M9 medium containing 0.5% D-glucose and 0.1% 4-[(phenylmethyl)oxy]butyric acid, followed by shaking the medium at 30° C. at 125 strokes/minute. At 48 hours later, cells were recovered by centrifugation, and they were resuspended in 200 mL of M9 medium, which contained 0.5% D-glucose and 0.1% 4-[(phenylmethyl)oxy]butyric acid but did not contain a nitrogen source (NH₄Cl), followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 26 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 90.5 mol % 3-hydroxy-4-[(phenylmethyl)oxy]butyric acid as a monomer unit.

### Example 15

A Pseudomonas cichorii YN2 strain was inoculated into 200 mL of M9 medium containing 0.5% D-glucose, 0.1% polypeptone, and 0.1% 4-[(phenylmethyl)oxy]butyric acid, followed by shaking the medium at 30° C. at 125 strokes/minute. At 48 hours later, 20 mL of aqueous solution containing 5% D-glucose and 1% 4-[(phenylmethyl)oxy]butyric acid was added thereto, followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 20 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 76.8 mol % 3-hydroxy-4-[(phenylmethyl)oxy]butyric acid as a monomer unit.

### Example 16

A Pseudomonas cichorii YN2 strain was inoculated into 200 mL of M9 medium containing 0.5% polypeptone and 0.1% 4-[(phenylmethyl)oxy]butyric acid, followed by shaking the medium at 30° C. at 125 strokes/minute. At 48 hours later, cells were recovered by centrifugation, and they were resuspended in 200 mL of M9 medium, which contained 0.5% pyruvic acid and 0.1% 4-[(phenylmethyl)oxy]butyric acid but did not contain a nitrogen source (NH₄Cl), followed by further shaking the resulting liquid at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 19 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 73.2 mol % 3-hydroxy-4-[(phenylmethyl)oxy]butyric acid as a monomer unit.

### Example 17

A Pseudomonas cichorii YN2 strain was inoculated into 200 mL of M9 medium containing 0.5% polypeptone and 0.1% 4-[(phenylmethyl)oxy]butyric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 15 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 76.7 mol % 3-hydroxy-4-[(phenylmethyl)oxy]butyric acid as a monomer unit.

### Example 18

A Pseudomonas cichorii H45 strain was inoculated into 200 mL of M9 medium containing 0.5% yeast extract and 0.1% 4-[(phenylmethyl)oxy]butyric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 14 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 75.5 mol % 3-hydroxy-4-[(phenylmethyl)oxy]butyric acid as a monomer unit.

### Example 19

A Pseudomonas jessenii P161 strain was inoculated into 200 mL of M9 medium containing 0.5% glucose and 0.1% 4-[(phenylmethyl)oxy]butyric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 11 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 85.9 mol % 3-hydroxy-4-[(phenylmethyl)oxy]butyric acid as a monomer unit.

### Example 20

A Pseudomonas cichorii YN2 strain was inoculated into 200 mL of M9 medium containing 0.5% pyruvic acid and 0.1% 4-[(phenylmethyl)oxy]butyric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 8 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 90.4 mol % 3-hydroxy-4-[(phenylmethyl)oxy]butyric acid as a monomer unit.

### Example 21

A Pseudomonas cichorii H45 strain was inoculated into 200 mL of M9 medium containing 0.5% sodium glutamate and 0.1% 4-[(phenylmethyl)oxy]butyric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 10 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 84.3 mol % 3-hydroxy-4-[(phenylmethyl)oxy]butyric acid as a monomer unit.

### Example 22

A Pseudomonas jessenii P161 strain was inoculated into 200 mL of M9 medium containing 0.1% nonanoic acid and 0.1% 4-[(phenylmethyl)oxy]butyric acid, followed by shaking the medium at 30° C. at 125 strokes/minute. At 48 hours later, cells were recovered by centrifugation and washed once with cold methanol, and then lyophilized.

The obtained lyophilized pellet was suspended in 20 mL of chloroform, and the suspension was stirred at 60° C. for 20 hours to extract PHA. The extract was filtered through a membrane filter with a pore size of 0.45 μm, and the filtrate was concentrated using a rotary evaporator. The obtained condensate was reprecipitated in cold methanol, and then only the precipitate was recovered and subjected to vacuum drying, to obtain 7 mg of PHA. As a result of carrying out an NMR analysis under the same conditions as in Example 1, it was found that the obtained PHA comprised 3-hydroxy-4-[(phenylmethyl)oxy]butyric acid as a monomer unit and further comprised, as a monomer unit, 3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid. Moreover, from the ¹H-NMR spectrum integration ratio, it was found that the obtained PHA comprised 21.9 mol % 3-hydroxy-4-[(phenylmethyl)oxy]butyric acid as a monomer unit.

### Example 23

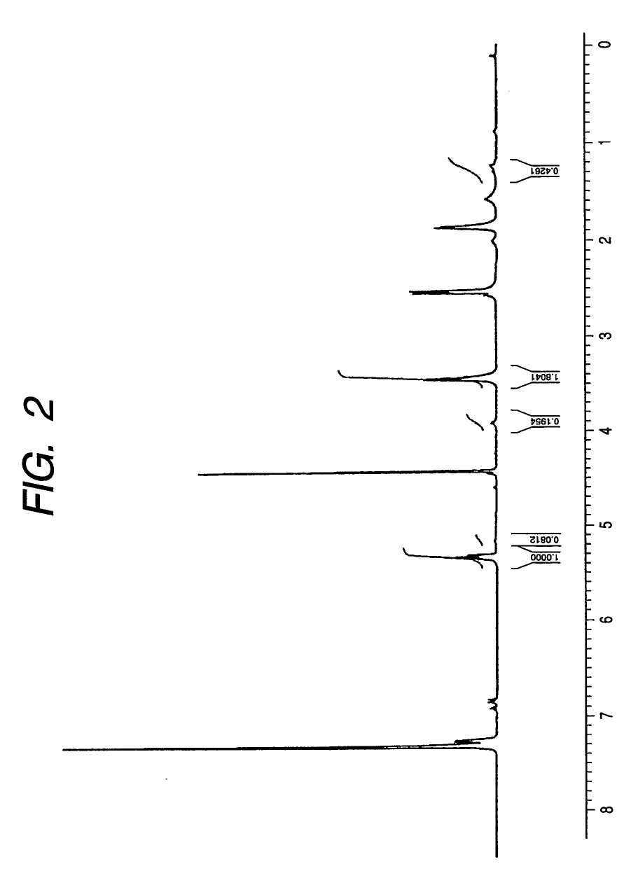

0.5% glucose, 6 mM 5-phenoxyvaleric acid, and 3 mM 5-[(phenylmethyl)oxy]valeric acid were dissolved in 100 ml of the above M9 medium, and the resultant solution was placed in a 200 ml shaking flask and was then sterilized with an autoclave, followed by cooling to room temperature. 2 ml of the culture solution of a Pseudomonas cichorii YN2 strain that had previously been subjected to shaking culture at 30° C. for 8 hours in an M9 medium containing 0.5% polypeptone was added to the above prepared medium, followed by culture at 30° C. for 48 hours. After completion of the culture, cells were recovered, and the thus obtained cells were resuspended in 100 ml of the same above medium, followed by culture in a 200 ml shaking flask at 30° C. for 42 hours. After completion of the culture, cells were recovered by centrifugation and washed with methanol, and then dried. After weighing the dried cells, chloroform was added thereto, and the mixture was stirred at 35° C. for 72 hours, to extract a polymer. The chloroform containing the extracted polymer was filtered, and the filtrate was concentrated using an evaporator. Thereafter, the precipitated and solidified portion was collected in cold methanol, and the portion was dried under reduced pressure, to obtain a polymer of interest. FIG. 2 shows the results obtained from an NMR analysis that was carried out under the same conditions as in Example 1. It was confirmed that the obtained PHA was a polyhydroxyalkanoate copolymer comprising the units expressed by the following chemical formula (27) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=63:37:0). Moreover, it was confirmed by ¹³C-NMR (<measuring equipment> FT-NMR: Bruker DP×400, resonance frequency: ¹³C=100 MHz, <measuring equipment> type of nuclear species: ¹³C, used solvent: CDCl₃, measuring temperature: room temperature) that the obtained PHA comprised unit B, that is, a 3-hydroxy-5-[(phenylmethyl)oxy]valeric acid unit.

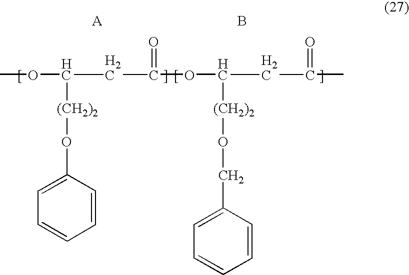

The molecular weight of the polymer was determined by gel permeation chromatography (GPC) (GPC: Tosoh HLC-8220, column: Tosoh TSK-GEL Super HM-H, solvent: chloroform, polystyrene conversion).

The weight of the obtained polymer (PDW) was 0.17 g/l, and the number average molecular weight of the obtained polymer was 93,000.

### Example 24

0.5% glucose, 0.1% polypeptone, 6 mM 5-phenoxyvaleric acid, and 3 mM 5-[(phenylmethyl)oxy]valeric acid were dissolved in 100 ml of the above M9 medium, and the resultant solution was placed in a 200 ml shaking flask and was then sterilized with an autoclave, followed by cooling to room temperature. 2 ml of the culture solution of a Pseudomonas cichorii YN2 strain that had previously been subjected to shaking culture at 30° C. for 8 hours in an M9 medium containing 0.5% polypeptone was added to the above prepared medium, followed by culture at 30° C. for 42 hours. After completion of the culture, cells were recovered and washed with methanol, and then dried. After weighing the dried cells, chloroform was added thereto, and the mixture was stirred at 35° C. for 72 hours, to extract a polymer. The chloroform containing the extracted polymer was filtered, and the filtrate was concentrated using an evaporator. Thereafter, the precipitated and solidified portion was collected in cold methanol, and the portion was dried under reduced pressure, to obtain a polymer of interest.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (27) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=38:33:29). Moreover, by ¹³C-NMR that was carried out under the same condition as in Example 23, it was confirmed that the obtained polymer comprised unit B, that is, a 3-hydroxy-5-[(phenylmethyl)oxy]valeric acid unit.

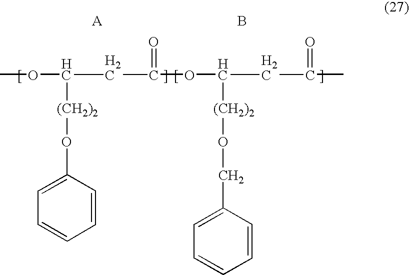

The molecular weight of the polymer was determined by GPC in the same manner as in Example 1.

The weight of the obtained polymer (PDW) was 0.06 g/l, and the number average molecular weight of the obtained polymer was 94,000.

### Example 25

A polymer of interest was obtained by the same method as in Example 24 with the exceptions that a Pseudomonas cichorii H45 strain was used instead of the YN2 strain used in Example 24, and that 0.5% yeast extract was used instead of glucose and polypeptone used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (27) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=42:33:25). Moreover, ¹³C-NMR was carried out in the same manner as in Example 23, and as a result, it was confirmed that the obtained polymer comprised unit B, that is, a 3-hydroxy-5-[(phenylmethyl)oxy]valeric acid unit.

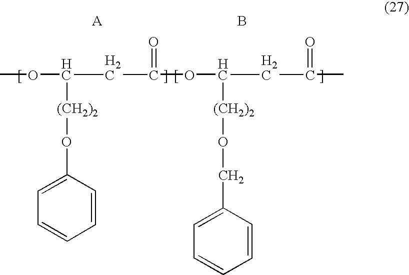

As with Example 1, the molecular weight of the obtained polymer was determined by GPC.

The weight of the obtained polymer (PDW) was 0.05 g/l, and the number average molecular weight of the obtained polymer was 91,000.

### Example 26

A polymer of interest was obtained by the same method as in Example 24 with the exceptions that a Pseudomonas cichorii H45 strain was used instead of the YN2 strain used in Example 24, and that 0.5% sodium pyruvate was used instead of glucose and polypeptone used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (27) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=58:24:18). Moreover, ¹³C-NMR was carried out in the same manner as in Example 23, and as a result, it was confirmed that the obtained polymer comprised unit B, that is, a 3-hydroxy-5-[(phenylmethyl)oxy]valeric acid unit.

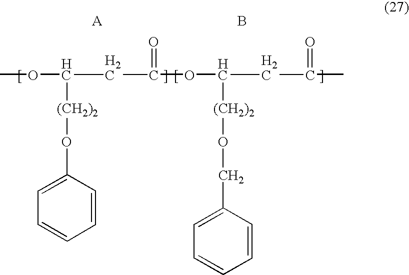

As with Example 1, the molecular weight of the obtained polymer was determined by GPC.

The weight of the obtained polymer (PDW) was 0.03 g/l, and the number average molecular weight of the obtained polymer was 102,000.

### Example 27

A polymer of interest was obtained by the same method as in Example 24 with the exceptions that a Pseudomonas jessenii P161 strain was used instead of the YN2 strain used in Example 24, and that 0.5% sodium glutamate was used instead of glucose and polypeptone used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (27) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=40:35:25). Moreover, ¹³C-NMR was carried out in the same manner as in Example 23, and as a result, it was confirmed that the obtained polymer comprised unit B, that is, a 3-hydroxy-5-[(phenylmethyl)oxy]valeric acid unit.

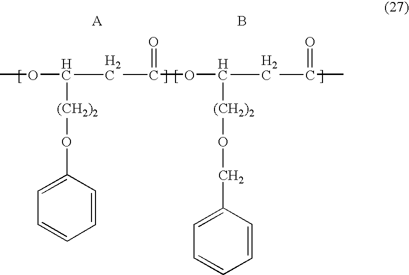

As with Example 1, the molecular weight of the obtained polymer was determined by GPC.

The weight of the obtained polymer (PDW) was 0.08 g/l, and the number average molecular weight of the obtained polymer was 89,000.

### Example 28

A polymer of interest was obtained by the same method as in Example 24 with the exceptions that a Pseudomonas jessenii P161 strain was used instead of the YN2 strain used in Example 24, and that 0.1% nonanoic acid was used instead of glucose and polypeptone used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (27) (A:B:other units=40:35:25). Moreover, ¹³C-NMR was carried out in the same manner as in Example 23, and as a result, it was confirmed that the obtained polymer comprised unit B, that is, a 3-hydroxy-5-[(phenylmethyl)oxy]valeric acid unit.

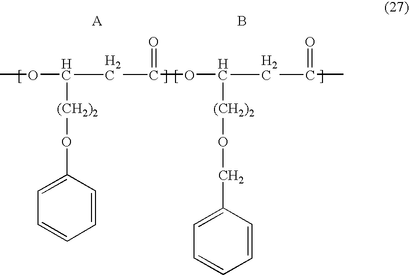

As with Example 1, the molecular weight of the obtained polymer was determined by GPC.

The weight of the obtained polymer (PDW) was 0.04 g/l, and the number average molecular weight of the obtained polymer was 98,000.

### Example 29

A polymer of interest was obtained by the same method as in Example 24 with the exception that 4-phenoxybutyric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (28) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=21:43:36). Moreover, ¹³C-NMR measurement was carried out in the same manner as in Example 23, and as a result, it was confirmed that the obtained polymer comprised unit B, that is, a 3-hydroxy-5-[(phenylmethyl)oxy]valeric acid unit.

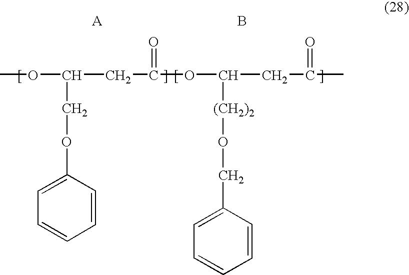

As with Example 1, the molecular weight of the obtained polymer was determined by GPC.

The weight of the obtained polymer (PDW) was 0.02 g/l, and the number average molecular weight of the obtained polymer was 92,000.

### Example 30

A polymer of interest was obtained by the same method as in Example 24 with the exception that 5-phenylvaleric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (29) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=56:25:19).

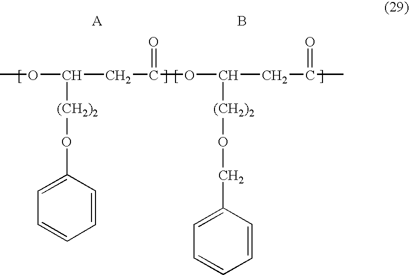

As with Example 1, the molecular weight of the obtained polymer was determined by GPC.

The weight of the obtained polymer (PDW) was 0.13 g/l, and the number average molecular weight of the obtained polymer was 98,000.

### Example 31

A polymer of interest was obtained by the same method as in Example 24 with the exception that 5-(4-vinylphenyl)valeric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (30) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=42:34:24).

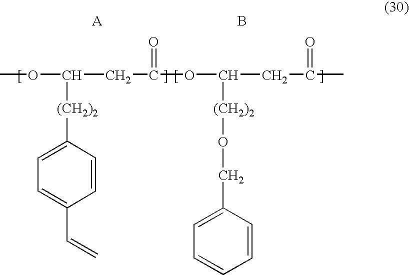

As with Example 1, the molecular weight of the obtained polymer was determined by GPC.

The weight of the obtained polymer (PDW) was 0.03 g/l, and the number average molecular weight of the obtained polymer was 87,000.

### Example 32

A polymer of interest was obtained by the same method as in Example 24 with the exception that 5-benzoylvaleric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (31) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=48:27:25).

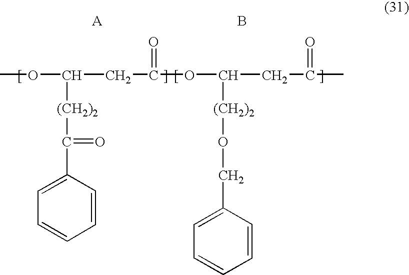

As with Example 1, the molecular weight of the obtained polymer was determined by GPC.

The weight of the obtained polymer (PDW) was 0.02 g/l, and the number average molecular weight of the obtained polymer was 160,000.

### Example 33

A polymer of interest was obtained by the same method as in Example 24 with the exception that 5-(phenylsulfanyl)valeric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (32) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=56:22:22).

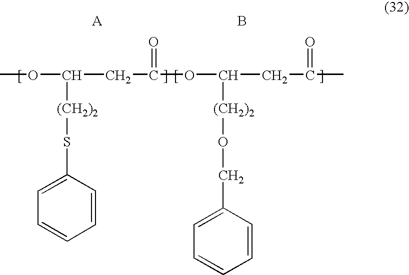

As with Example 1, the molecular weight of the obtained polymer was determined by GPC.

The weight of the obtained polymer (PDW) was 0.11 g/l, and the number average molecular weight of the obtained polymer was 89,000.

### Example 34

A polymer of interest was obtained by the same method as in Example 24 with the exception that 5-[(phenylmethyl)sulfanyl]valeric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (33) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=46:31:24).

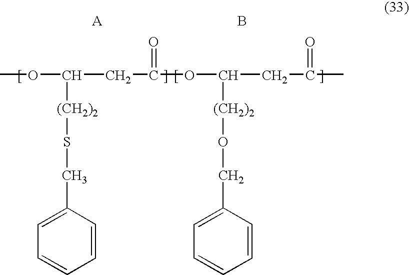

As with Example 1, the molecular weight of the obtained polymer was determined by GPC.

The weight of the obtained polymer (PDW) was 0.04 g/l, and the number average molecular weight of the obtained polymer was 84,000.

### Example 35

A polymer of interest was obtained by the same method as in Example 24 with the exception that 5-(2-thienyl)valeric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (34) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=51:26:23).

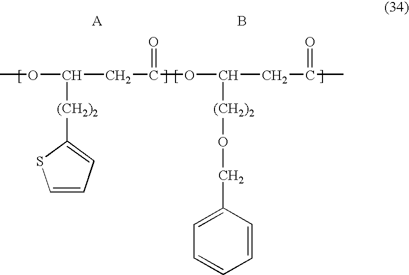

As with Example 1, the molecular weight of the obtained polymer was determined by GPC.

The weight of the obtained polymer (PDW) was 0.09 g/l, and the number average molecular weight of the obtained polymer was 86,000.

### Example 36

A polymer of interest was obtained by the same method as in Example 24 with the exception that 5-(2-thienylsulfanyl)valeric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (35) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=49:40:11).

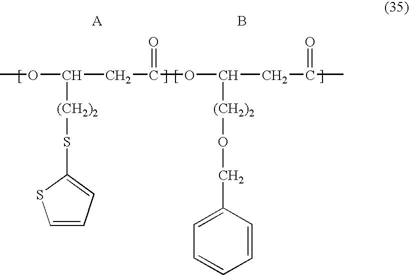

As with Example 1, the molecular weight of the obtained polymer was determined by GPC.

The weight of the obtained polymer (PDW) was 0.10 g/l, and the number average molecular weight of the obtained polymer was 81,000.

### Example 37

A polymer of interest was obtained by the same method as in Example 24 with the exception that 5-(2-thienylcarbonyl)valeric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (36) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=41:40:19).

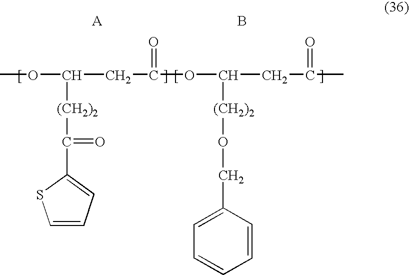

As with Example 1, the molecular weight of the obtained polymer was determined by GPC.

The weight of the obtained polymer (PDW) was 0.02 g/l, and the number average molecular weight of the obtained polymer was 89,000.

### Example 38

A polymer of interest was obtained by the same method as in Example 24 with the exception that 5-cyclohexylvaleric acid was used instead of 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (37) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=46:28:26).

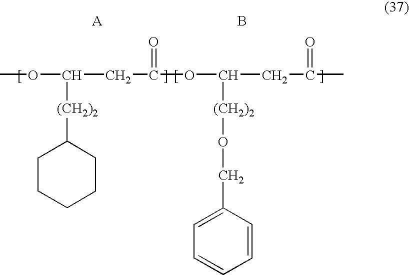

As with Example 1, the molecular weight of the obtained polymer was determined by GPC.

The weight of the obtained polymer (PDW) was 0.08 g/l, and the number average molecular weight of the obtained polymer was 92,000.

### Example 39

A polymer of interest was obtained by the same method as in Example 24 with the exception that 4-[(phenylmethyl)oxy]butyric acid was used instead of 5-[(phenylmethyl)oxy]valeric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (38) (A:B:other units (3-hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=49:24:27). Moreover, ¹³C-NMR measurement was carried out in the same manner as in Example 23, and as a result, it was confirmed that the obtained polymer comprised unit B, that is, a 3-hydroxy-4-[(phenylmethyl)oxy]butyric acid unit.

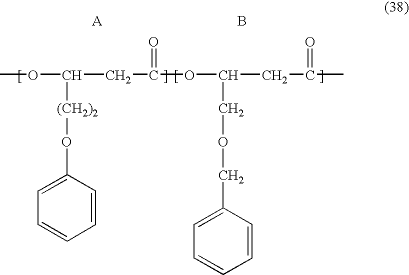

As with Example 1, the molecular weight of the obtained polymer was determined by GPC.

The weight of the obtained polymer (PDW) was 0.02 g/l, and the number average molecular weight of the obtained polymer was 91,000.

### Example 40

A polymer of interest was obtained by the same method as in Example 24 with the exception that 3 mM 5-phenoxyvaleric acid and 3 mM 5-cyclohexylvaleric acid were used instead of 6 mM 5-phenoxyvaleric acid used in Example 24.

To determine the structure of the obtained polymer, ¹H-NMR was carried out in the same manner as in Example 1. As a result, it was confirmed that the obtained polymer was a polyhydroxyalkanoate copolymer comprising the units expressed by the following chemical formula (39) (A:B:C:other units (3hydroxyalkanoic acid or 3-hydroxyalkenoic acid, which is saturated or unsaturated fatty acid having 4 to 12 carbon atoms, such as 3-hydroxybutyric acid or 3-hydroxyvaleric acid)=31:28:21:20). Moreover, ¹³C-NMR measurement was carried out in the same manner as in Example 23, and as a result, it was confirmed that the obtained polymer comprised unit C, that is, a 3-hydroxy-5-[(phenylmethyl)oxy]valeric acid unit.

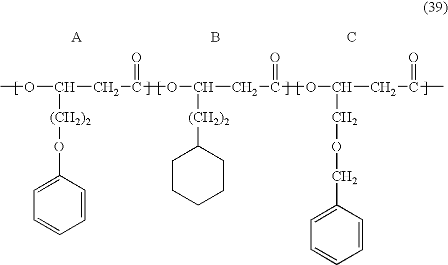

As with Example 1, the molecular weight of the obtained polymer was determined by GPC.

The weight of the obtained polymer (PDW) was 0.09 g/l, and the number average molecular weight of the obtained polymer was 93,000.

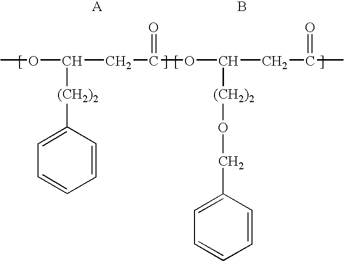

1. A polyhydroxyalkanoate comprising a 3-hydroxy-ω-[(phenylmethyl)oxy]alkanoic acid unit expressed by chemical formula (1): 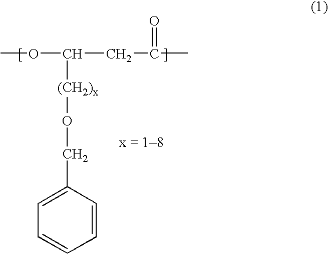
wherein x can be one or more integers within the range shown in the chemical formula.

2. The polyhydroxyalkanoate according to claim 1, comprising at least one unit expressed by chemical formula selected from the group consisting of chemical formulas (2) and (3): 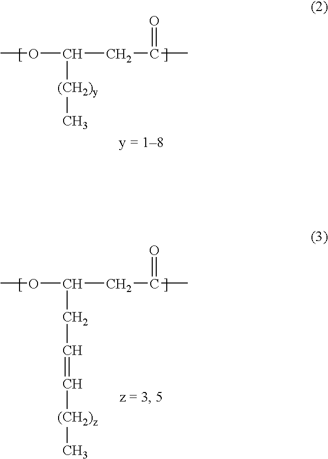
wherein y and z can be one or more integers within the range shown in the chemical formulas, while being independent from the unit expressed by chemical formula (1).

3. The polyhydroxyalkanoate according to claim 1, comprising, in a molecule thereof, the 3-hydroxy-ω-[(phenylmethyl)oxy]alkanoic acid unit expressed by chemical formula (1) and a 3-hydroxy-alkanoic acid unit expressed by chemical formula (4): 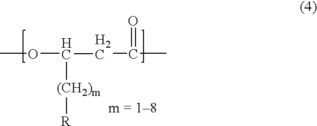
wherein m can be one or more integers within the range shown in the chemical formula, and R comprises a residue having either a phenyl structure or thienyl structure, or a 3-hydroxy-ω-cyclohexylalkanoic acid unit expressed by chemical formula (5): 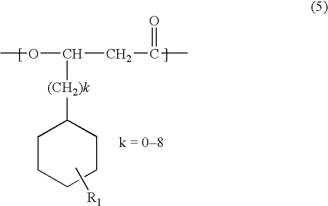
wherein R₁ is H, CN, NO₂, halogen, CH₃, C₂H₅, C₃H₇, CF₃, C₂F₅ and C₃F₇, and k can be one or more integers within the range shown in the chemical formula.

4. The polyhydroxyalkanoate according to claim 1, wherein the 3-hydroxy-ω-[(phenylmethyl)oxy]alkanoic acid unit expressed by chemical formula (1) is either one or both of:
a 3-hydroxy-4-[(phenylmethyl)oxy]butyric acid unit expressed by chemical formula (6): 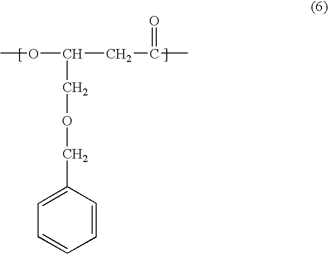
and a 3-hydroxy-5-[(phenylmethyl)oxy]valeric acid unit expressed by chemical formula (7):

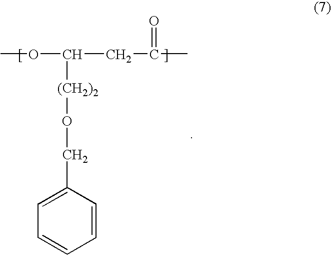

5. The polyhydroxyalkanoate according to claim 3, wherein R in chemical formula (4) is a group selected from the group consisting of 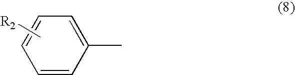
wherein R₂ is H, halogen, CN, NO₂, CH₃, C₂H₅, C₃H₇, CH═CH₂, COOR₃ (wherein R₃ represents any one selected from the group consisting of H, Na and K), CF₃, C₂F₅ and C₃F₇, and in a case where there exist a plurality of units, R₂ may be different for each unit; 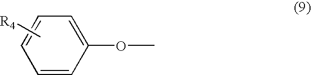
wherein R₄ is selected from the group consisting of H, halogen, CN, NO₂, CH₃, C₂H₅, C₃H₇, SCH₃, CF₃, C₂F₅ and C₃F₇, and in a case where there exist a plurality of units, R₄ may be different for each unit; 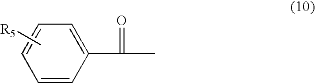
wherein R₅ is selected from the group consisting of H, halogen, CN, NO₂, CH₃, C₂H₅, C₃H₇, CF₃, C₂F₅ and C₃F₇, and in a case where there exist a plurality of units, R₅ may be different for each unit; 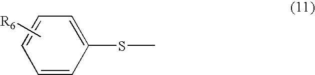
wherein R₆ is selected from the group consisting of H, halogen, CN, NO₂, COOR₇, SO₂R₈ (wherein R₇ represents any one selected from the group consisting of H, Na, K, CH₃ and C₂H₅, and Re represents any one selected from the group consisting of OH, ONa, OK, halogen, OCH₃ and OC₂H₅), CH₃, C₂H₅, C₃H₇, (CH₃)₂—CH, and (CH₃)₃—C, and in a case where there exist a plurality of units, R₆ may be different for each unit; 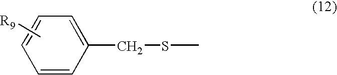
wherein R₉ represents a substituent group on the aromatic ring, R₉ is selected from thg group consisting of H, halogen, CN, NO₂, COOR₁₀, SO₂R₁₁ (wherein R₁₀ represents any one selected from the group consisting of H, Na, K, CH₃ and C₂H₅, and R₁₁ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₉ may be different for each unit; 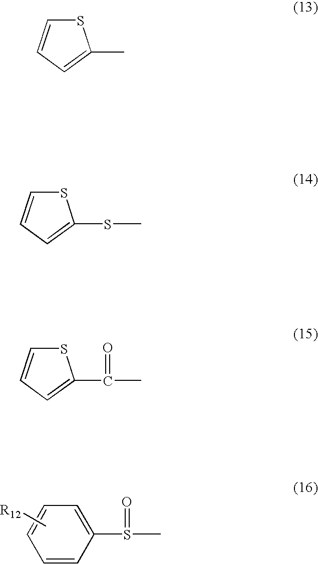
wherein R₁₂ is selected from thg group consisting of H, halogen, CN, NO₂, COOR₁₃, SO₂R₁₄ (wherein R₁₃ represents any one selected from the group consisting of H, Na, K, CH₃ and C₂H₅, and R₁₄ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₁₂ may be different for each unit; and 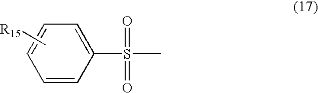
wherein R₁₅ is selected from the group consisting of H, halogen, CN, NO₂, COOR₁₆, SO₂R₁₇ (wherein R₁₆ represents any one selected from the group consisting of H, Na, K, CH₃ and C₂H₅, and R₁₇ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₁₅ may be different for each unit.

6. The polyhydroxyalkanoate according to claim 1, wherein a number average molecular weight is within the range between 1,000 and 1,000,000.

7. A method for producing a polyhydroxyalkanoate comprising, in a molecule thereof, a 3-hydroxy-ω-[(phenylmethyl)oxy]alkanoic acid unit expressed by chemical formula (1): 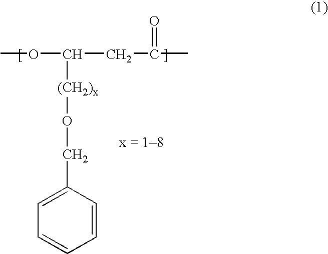
wherein x can be one or more integers within the range shown in the chemical formula, which comprises allowing a microorganism with an ability to produce a polyhydroxyalkanoate comprising in a molecule thereof a 3-hydroxy-ω-[(phenylmethyl)oxy]alkanoic acid unit expressed by chemical formula (1) to biosynthesize the polyhydroxyalkanoate under a condition which comprise ω-[(phenylmethyl)oxy]alkanoic acid expressed by chemical formula (19): 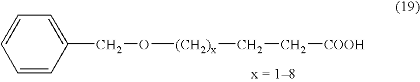
wherein x can be one or more integers within the range shown in the chemical formula.

8. The method for producing a polyhydroxyalkanoate according to claim 7, wherein the polyhydroxyalkanoate comprises at least one unit expressed by the following chemical formulas (2) and (3): 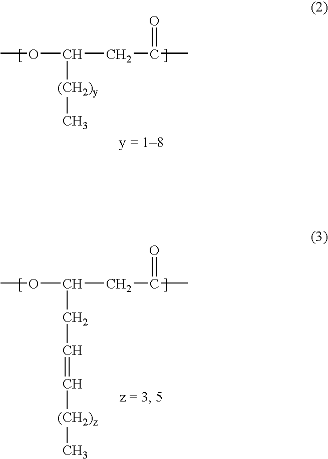
wherein y and z can be one or more integers within the range shown in the chemical formulas, while being independent from the unit expressed by chemical formula (1).

9. The method for producing a polyhydroxyalkanoate according to claim 7, comprising allowing the microorganism with an ability to produce a polyhydroxyalkanoate comprising, in a molecule thereof, the 3-hydroxy-ω-[(phenylmethyl)oxy]alkanoic acid unit expressed by chemical formula (1) and a 3-hydroxy-alkanoic acid unit expressed by chemical formula (22): 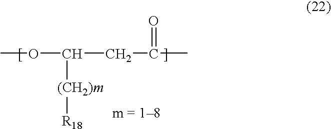
wherein m can be one or more integers within the range shown in the chemical formula, and R₁₈ comprises a residue having either a phenyl structure or thienyl structure, or 3-hydroxy-ω-cyclohexylalkanoic acid unit expressed by chemical formula (5): 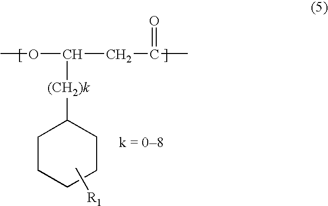
wherein R₁ is selected from the group consisting of H, CN, NO₂, halogen, CH₃, C₂H₅, C₃H₇, CF₃, C₂F₅ and C₃F₇, and k can be one or more integers within the range shown in the chemical formula,
from ω-[(phenylmethyl)oxy]alkanoic acid expressed by chemical formula (19), and a alkanoic acid expressed by chemical formula (20): 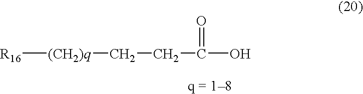
wherein q can be one or more integers within the range shown in the chemical formula, and R₁₆ comprises a residue having either a phenyl structure or thienyl structure, or ω-cyclohexylalkanoic acid expressed by chemical formula (21): 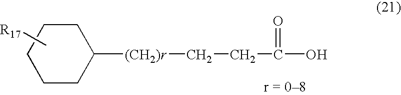
wherein R₁₇ is selected from the group consisting of H, CN, NO₂, halogen, CH₃, C₂H₅, C₃H₇, CF₃, C₂F₅ and C₃F₇, and r can be one or more integers within the range shown in the chemical formula as raw materials to biosynthesize the polyhydroxyalkanoate under a condition which comprise ω-[(phenylmethyl)oxy]alkanoic acid expressed by chemical formula (19), and alkanoic acid expressed by chemical formula (20) or ω-cyclohexylalkanoic acid expressed by chemical formula (21).

10. The method for producing a polyhydroxyalkanoate according to claim 7, wherein the ω-[(phenylmethyl)oxy]alkanoic acid expressed by said chemical formula (19) is 4-[(phenylmethyl)oxy]butyric acid expressed by chemical formula (23): 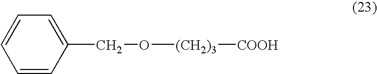
or 5-[(phenylmethyl)oxy]valeric acid expressed by chemical formula (24):

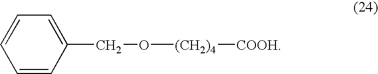

11. The method for producing a polyhydroxyalkanoate according to claim 9, wherein R₁₆ in chemical formula (20) and R₁₈ in chemical formula (22) are groups independently selected from the group consisting of 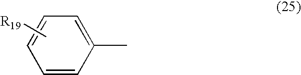
wherein R₁₉ is selected from the group consisting of H, halogen, CN, NO₂, CH₃, C₂H₅, C₃H₇, CH═CH₂, CF₃, C₂F₅ and C₃F₇, and in a case where there exist a plurality of units, R₁₉ may be different for each unit; 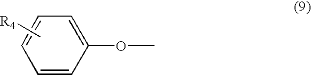
wherein R₄ is selected from the group consisting of H, halogen, CN, NO₂, CH₃, C₂H₅, C₃H₇, SCH₃, CF₃, C₂F₅ and C₃F₇, and in a case where there exist a plurality of units, R₄ may be different for each unit; 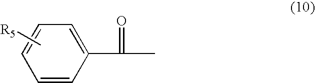
wherein R₅ is selected from the group consisting of H, halogen, CN, NO₂, CH₃, C₂H₅, C₃H₇, CF₃, C₂F₅ and C₃F₇, and in a case where there exist a plurality of units, R₅ may be different for each unit; 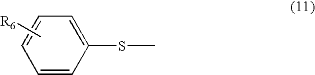
wherein R₆ is selected from the group consisting of H, halogen, CN, NO₂, COOR₇, SO₂R₈ (wherein R₇ represents any one selected from the group consisting of H, Na, K, CH₃ and C₂H₅, and R₈ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₆ may be different for each unit; 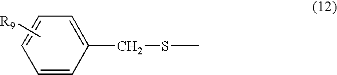
wherein R₉ is selected from the group consisting of H, halogen, CN, NO₂, COOR₁, SO₂R₁₁ (wherein R₁₀ represents any one selected from the group consisting of H, Na, K, CH₃ and C₂H₅, and R₁₁ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₉ may be different for each unit; 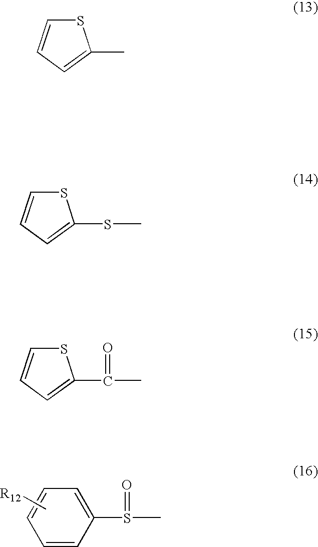
wherein R₁₂ is selected from the group consisting of H, halogen, CN, NO₂, COOR₁₃, SO₂R₁₄ (wherein R₁₃ represents any one selected from the group consisting of H, Na, K, CH₃ and C₂H₅, and R₁₄ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₁₂ may be different for each unit; and 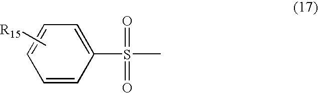
wherein R₁₅ is selected from the group consisting of H, halogen, CN, NO₂, COOR₁₆, SO₂R₁₇ (wherein R₁₆ represents any one selected from the group consisting of H, Na, K, CH₃ and C₂H₅, and R₁₇ represents any one selected from the group consisting of OH, ONa, OK, halogen, OCH₃ and OC₂H₅), CH₃, C₂H₅, C₃H₇, (CH₃)₂—CH and (CH₃)₃—C, and in a case where there exist a plurality of units, R₁₅ may be different for each unit.

12. The method for producing a polyhydroxyalkanoate according to claim 7, wherein said condition is that said microorganisms is cultured in a medium containing ω-[(phenylmethyl)oxy]alkanoic acid expressed by chemical formula (19).

13. The method for producing a polyhydroxyalkanoate according to claim 9, wherein said condition is that said microorganism is cultured in a medium containing the ω-[(phenylmethyl)oxy]alkanoic acid expressed by chemical formula (19) and the alkanoic acid expressed by chemical formula (20) or the ω-cyclohexylalkanoic acid expressed by chemical formula (21).

14. The method for producing a polyhydroxyalkanoate according to claim 12, wherein said medium contains at least one selected from the group consisting of peptides, yeast extract, organic acids or salts thereof, amino acids or salts thereof, saccharides and straight-chain alkanoic acids, which is saturated or unsaturated fatty acid having 4 to 12 carbon atoms or salts thereof.

15. The method for producing a polyhydroxyalkanoate according to claim 14, wherein the peptide is polypeptone; the organic acids or salts thereof are one or more compounds selected from the group consisting of pyruvic acid, oxaloacetic acid, citric acid, isocitric acid, ketoglutaric acid, succinic acid, fumaric acid, malic acid, lactic acid, and salts thereof; the amino acids or salts thereof are one or more compounds selected from the group consisting of glutamic acid, aspartic acid, and salts thereof; and the saccharides are one or more compounds selected from the group consisting of glyceroaldehyde, erythrose, arabinose, xylose, glucose, galactose, mannose, fructose, glycerol, erythritol, xylitol, gluconic acid, glucuronic acid and galacturonic acid, maltose, sucrose and lactose.

16. The method for producing a polyhydroxyalkanoate according to claim 12, wherein said culture of microorganisms comprises two or more culturing steps.

17. The method for producing a polyhydroxyalkanoate according to claim 16, wherein said culture is a fed-batch culture.

18. The method for producing a polyhydroxyalkanoate according to any one of claims 12 to 17, comprising a step of recovering a polyhydroxyalkanoate comprising 3-hydroxy-ω-[(phenylmethyl)oxy]alkanoic acid unit expressed by chemical formula (1) generated by the microorganism from the cells of the microorganism.

19. The method for producing a polyhydroxyalkanoate according to claim 7, wherein said microorganism belongs to Pseudomonas species.

20. The method for producing a polyhydroxyalkanoate according to claim 19, wherein said microorganism is one or more strains selected from the group consisting of Pseudomonas cichorii YN2 (FERM BP-7375), Pseudomonas cichorii H45 (FERM BP-7374) and Pseudomonas jessenii P161 (FERN BP-7376).
